(19) United States
(12) Patent Application Publication
(10) Pub. No.: US 2021/0308388 A1
(43) Pub. Date: Oct. 7, 2021

(54) VAPORIZER AND METHOD OF OPERATING A VAPORIZER

(71) Applicant: Steven R. Vosen, Alameda, CA (US)

(72) Inventor: Steven R. Vosen, Alameda, CA (US)

(21) Appl. No.: 17/287,796

(22) PCT Filed: Oct. 24, 2019
(86) PCT No.: PCT/US19/57918

§ 371 Date: Apr. 22, 2021

Related U.S. Application Data
(60) Provisional application No. 62/750,020, filed on Oct. 24, 2018.

(51) Int. Cl. A61M 11/04

(52) U.S. Cl. CPC A61M 11/042; A61M 2205/3368; A61M 2205/583; A61M 2205/582; A61M 2205/581

(57) ABSTRACT

A vaporizer and method of operating a vaporizer are described. The vaporizer includes a sensor for monitoring the state of vaporization. The method includes determining the state of the material being vaporized and turning off the vaporizer and/or signaling a user when the material is sufficiently vaporized.

### TECHNICAL FIELD

The present invention generally relates to vaporizers, and more particularly to a method and system for monitoring the state of a vaporizing material.

### BACKGROUND ART

Combustion is used to produce vapors from certain materials which may are inhaled for medicinal purpose, for aromatherapy, or for the ingestion of tobacco or other smoked substances. A major problem with combustion is that the vapors include combustion products which are poisonous, carcinogenic, or have unwanted side effects.

Vaporization without combustion is also used to produce vapors from materials. In vaporization, material is heated to a temperature sufficient to vaporize at least some of the material without igniting the material. Vaporization has many benefits over combustion, including the absence of combustion products, and providing vapors at a much lower temperature, both of which makes it much easier to inhale the vapors.

One disadvantage of vaporization of certain materials, such as plant material, is that it may be difficult to determine when there is no longer any material left to be vaporized. Thus, for example, when the material includes components that are not vaporized, the only indication that the active ingredients have been vaporized may be a slight change in color of the material.

Thus, there is a need in the art for a method and apparatus that permits the user of a vaporizer to easily determine of all, or most, of the active ingredients have been vaporized. Such a method and apparatus should be accurate, inexpensive, and easy to implement.

### DISCLOSURE OF INVENTION

Certain embodiments provide an apparatus for vaporizing a compound of a material. The apparatus includes: a receptacle for accepting the material; a heating element operational for heating the accepted material to a temperature sufficient to vaporize the compound; and electronics configured to determine when the accepted material is sufficiently vaporized.

Certain other embodiments provide an apparatus for vaporizing a compound of a material. The apparatus includes: a receptacle for accepting the material; a heating element adapted to heat the accepted material to a temperature sufficient to vaporize the compound; and electronics including one or more temperature sensors and power to operate the heating element. The electronics are configured to turn the heater off according to the output of at least one of the one or more temperature sensors.

Certain embodiments provide a method of operating an apparatus for vaporizing a compound of a material. The apparatus includes a receptacle for accepting the material and a heater to heat the accepted material to a temperature sufficient to vaporize the compound. The method includes estimating if the accepted material is sufficiently vaporized; and turning off the heater if the accepted material is sufficiently vaporized.

Certain other embodiments provide a method of operating an apparatus for vaporizing a compound of a material. The apparatus includes an air inlet to provide air to a receptacle for accepting the material, a heater to heat the accepted material to a temperature sufficient to vaporize the compound, a mouthpiece to accept air and vapors from the receptacle, and a temperature sensor. The method includes turning off the heater when the temperature measured by the temperature sensor equals or is greater than a first temperature.

These features together with the various ancillary provisions and features which will become apparent to those skilled in the art from the following detailed description, are attained by the apparatus and method of operating an apparatus of the present invention, preferred embodiments thereof being shown with reference to the accompanying drawings, by way of example only, wherein:

### BRIEF DESCRIPTION OF DRAWINGS

FIG. 1 is a side view of a first embodiment vaporizer of the present invention;

FIG. 2 is a top view of a the vaporizer of FIG. 1;

FIG. 3 is a sectional view of a second embodiment vaporizer 3-3 of FIG. 2;

FIG. 4 is a detailed view 4-4 of FIG. 2;

FIG. 5 is a detailed view 5-5 of a third embodiment vaporizer; and

FIG. 6 is a schematic diagram of the electronics of a vaporizer;

Reference symbols are used in the Figures to indicate certain components, aspects or features shown therein, with reference symbols common to more than one Figure indicating like components, aspects or features shown therein.

### MODE(S) FOR CARRYING OUT THE INVENTION

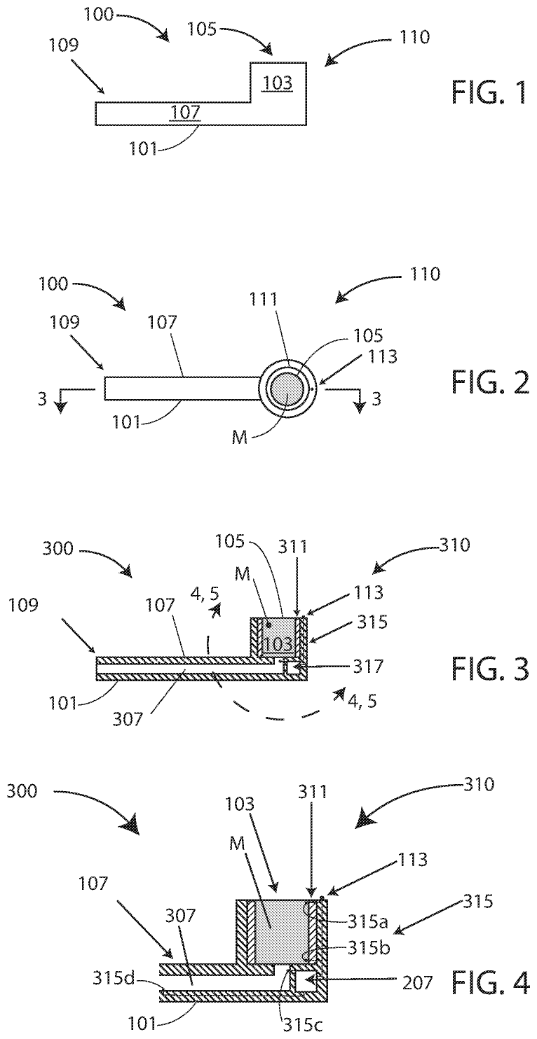

FIGS. 1 and 2 are a side view and top view, respectively, of a first embodiment vaporizer 100. Vaporizer 100 includes a housing 101 that contains a receptacle 103 having an open top 105 and into which a material M may be provided, a stem 107, a mouthpiece 109, and a heating system 110. As discussed subsequently, vaporizer 100 has an internal passageway that connects top 105 to mouthpiece 109, and heating system 110 includes a heating element 111, a switch 112, and other elements described subsequently including an optional signaling device 113.

Vaporizer 100 is configured to heat material M sufficient to vaporize at least some of the material M may be, for example, be a solid and/or liquid material at room temperature that is at least partially converted to a gas (also referred to as a vapor) at a sufficiently high temperature. Certain materials, especially plant materials, may include a combination of materials including some that that vaporize at some temperature (a “vaporization temperature”) to produce useful vapors and others that will ignite at temperature higher than the vaporization temperature. Thus, for example, material M may include material Mv which is converted from a solid or liquid to a vapor at a vaporization temperature, and material Mu which remains after material Mv is vaporized.

As discussed subsequently in greater detail, material M maybe provided to receptacle 103, heating system 110 may be activated using switch 112, and a user may inhale through mouthpiece 109, drawing air through top 105 and over the heated material M. The air and/or material are heated by heating system 110, vaporizing material Mv material, which mixes with air drawn in through top 105. In certain embodiments, as discussed subsequently, heating system 110 determines when vaporization is complete and turns off the heating system. In other certain embodiments, heating system 110 actuates optional signaling device 113 to provide the user with a visible or auditory signal that vaporization is complete.

In certain embodiments, switch 112 is a button or other touch sensor, or is operated by a flow sensor that determines when the user is inhaling on mouthpiece 109.

In various embodiments, heating element 111 is a resistance heater or an active heating element, such as a heater operating using the Peltier effect.

In various other embodiments, signaling device 113 is one or more of: a light which provides a visual signal; a speaker which provides an auditory signal; and/or a vibrating element to provide a tactile signal.

FIG. 3 is a sectional view 3-3 of a second embodiment vaporizer 300 and FIG. 4 is a detailed view 4-4 of FIG. 3. Vaporizer 300 is generally similar to vaporizer 100, except as explicitly noted below, and includes a heating system 310 that is generally similar to heating system 110, except as explicitly noted below.

Stem 107 of vaporizer 300 includes an internal passageway 307 that that provides fluid communication between receptacle 103 and mouthpiece 109. Heating system 110 of vaporizer 300 includes electronics 317 which accepts signals from one or more sensors 315 and provides electric power to heating element 311, which may be for example and without limitation a resistance heater, and which activates optional signaling device 113.

As illustrated in FIG. 4, one or more sensors 315 include one or more of a sensor 315a, 315b, 315c, and/or 315d. Sensors 315 include, in various embodiments, temperature sensors, including but not limited to thermocouples, and/or heat flux sensors which are within housing 101 or which protrude into a gas flow through the vaporizer. As an example of exemplary placement of sensors 315, sensor 315a is shown at or near top 105, sensor 315b is shown within and near the bottom of receptacle 103, sensor 315c is shown below the receptacle, and sensor 315d is shown within stem 107.

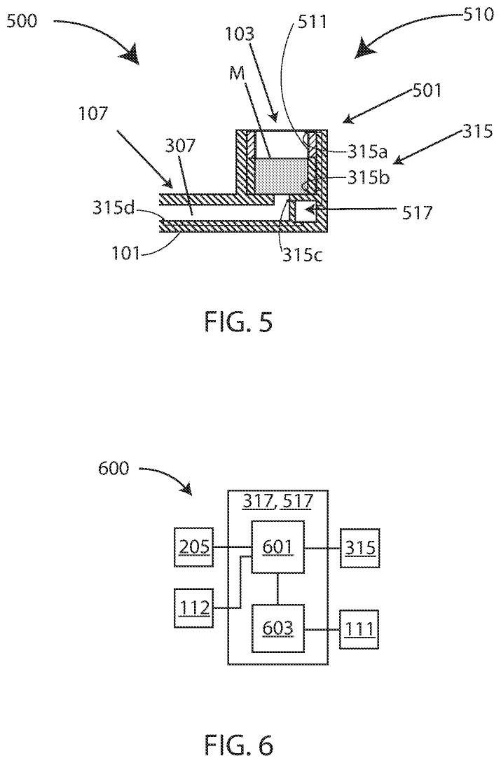

FIG. 5 is a detailed sectional view 5-5 of FIG. 3, illustrating a third embodiment vaporizer 500. Vaporizer 500 is generally similar to vaporizer 100 or 300, except as explicitly noted below, and includes a heating system 510 that is generally similar to heating system 110 or 310, except as explicitly noted below.

Vaporizer 500 includes an air preheat chamber 501 disposed between top 105 and material M, and which is surrounded by heating element 511. Air preheat chamber 501 is configured to heat air entering vaporizer 500 before the air flows through material M. Heating system 510 includes electronics 517 which accepts signals from one or more sensors 315 and provides electric power to heating element 511.

FIG. 6 is a schematic diagram 600 illustrating the control of vaporizer 100. Electronics 600, for example and without limitation, includes a processor 601, switch 112, and an electric power source 603. Processor 601 includes programming to direct electricity from power source 602 to heating element 111. Processor 601 further includes programming to accept signals from sensors 315 and determine if material M is sufficiently vaporized. If the programming in processor 601 determines that material M is not sufficiently vaporized, the programming directs the continued powering of heating element 111. If the programming in processor 601 determines that material M is sufficiently vaporized, then the programming in processor 601 provides instructions to electric power source 603 to stop providing power to heating element 111 and/or to actuate signaling device 113.

Vaporizer 100 may thus be operated as follows. Material M is provided to receptacle 113 and the user activates switch 112 by pressing a button, or alternatively by inhaling on mouthpiece 109. Electronics 600 monitor the output of one or more sensors 315 and provides power from power source 602 to heating element 111. Electronics 600 continues to power heating element 111 until the programming of processor 601 determines that material Mv is sufficiently vaporized. At this point, electronics 600 stops providing power to heating element 111 and/or activates signaling device 113.

Processor 601 may thus be programmed to power vaporizer 100 according to the output of sensors 315 to turn off the vaporizer and/or signal the user when it is determined that material M is sufficiently vaporized.

In certain embodiments, the programming of processor 601 is determined experimentally from a correlation of the temporal evolution of sensor measurements and an independent measure of the degree of vaporization. Thus, for example, a measurement of the output of sensor 315b, or a comparison of the output of sensors 315a and 315b, and an independent analytical determination of nicotine (for the example of M being tobacco) will determine sensor outputs corresponding to the complete vaporization of the tobacco. This data can then be programmed into processor 601 to control heating system 110, to stop the power to heating element 111 and/or to actuate optional signaling device 113 when vaporization is complete.

In certain embodiments, processor 601 determines that sufficient vaporization is achieved when a temperature, Tₛ, of one or more of sensors 315 downstream of the vaporization (such a sensor 315b, 315c, or 315d) exceeds a known or independently determined set temperature, T*, that is when Tₛ>T*.

In various embodiments, the value of T* may be: 1) the vaporization temperature, Tᵥ, of a specific component of material Mv; 2) a value greater than Tᵥ; or 3) an experimentally determined number based on the operation of vaporizer 100, which may also depend on the location of sensor 315 within vaporizer 100. In other embodiments, the variation in time of Tₛ(t) is utilized to, for example, determine if the operation of vaporizer 100 is changing over time due to, for example, deposits that may build up on heating element 111 or sensor 315, and correct a value of T*. In yet other embodiments, a phenomenological model of vaporization may be programmed into processor 401 which utilizes either Tₛ or Tₛ(t).

In certain other embodiments, processor 601 determines if sufficient vaporization is achieved based on the temporal rate of change of temperature, Tₛ(t), of one or more of sensors 315 downstream of the vaporization (such a sensor 315b, 315c, or 315d). Thus, for example, an increase in temperature at sensor 315b is indicative a reduced vaporization of M, and thus provides a basis for turning off power to heating element 111. Processor 601 may thus be programmed to determine the rate of change of the temperature of sensor 315b and to turn off power to heating element 111 when the rate of changes increases to some predetermined value.

In certain other embodiments, processor 601 determines if sufficient vaporization is achieved from the difference in output between of two sensors, such as the difference in temperature as measured by sensor 315b and the temperature measured by sensor 315a. Processor 601 may thus be programmed to determine the temperature difference noted above and to turn off power to heating element 111 when the rate of changes increases to some predetermined value.

In certain embodiments, processor 601 determines if sufficient vaporization is achieved from a combination of one or more sensor outputs, differences in sensor outputs, time derivative of censor outputs, or known or determined vaporization temperatures.

In certain other embodiments, Tₛ(t) is used to determine a heat flux through a surface in the vaporizer for additional information on the operation of vaporizer 100. In yet other embodiments, processor 601 utilizes combinations of measurements of sensors 315 to correct for ambient temperature, such as sensor 315a to determine vaporization of material M or an estimate of the amount of vapor being vaporized.

In addition, single or multiple measurements, in time or space, may be used to estimate properties of the air and vapor flowing from the receptacle that are indicative of the amount of vapor flowing to mouthpiece 109. Thus, for example, physical properties, such as the heat capacity of the vapor and air can be determined from various temperature measurements, which can also provide an indication of how vaporized material M is.

In other embodiments, programming in processor 601 operates heater 110 to vary the heating power over time. Phenomenological models of unsteady heat transfer in processor 601 may combine the unsteady heating of material M with the temporal sensor(s) output to determine the state of the vaporized material.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

Similarly, it should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

It is to be understood that the invention includes all of the different combinations embodied herein. Throughout this specification, the term “comprising” shall be synonymous with “including,” “containing,” or “characterized by,” is inclusive or open-ended and does not exclude additional, unrecited elements or method steps. “Comprising” is a term of art which means that the named elements are essential, but other elements may be added and still form a construct within the scope of the statement. “Comprising” leaves open for the inclusion of unspecified ingredients even in major amounts.

Thus, while there has been described what is believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

1. An apparatus for vaporizing a compound of a material, said apparatus comprising:
a receptacle for accepting the material;
a heating element operational for heating the accepted material to a temperature sufficient to vaporize the compound; and
electronics configured to determine when the accepted material is completely vaporized.

2. The apparatus of claim 1, where said electronics include one or more sensors to measure a state of the apparatus, and where said electronics determine when the accepted material is completely vaporized from the measured state.

3. The apparatus of claim 2, where said one or more sensors include a temperature sensor, and where said electronics determine when the accepted material is completely vaporized from an output of said temperature sensor.

4. The apparatus of claim 2, where said sensor includes a first temperature sensor and a second temperature sensor, and where said electronics determine when the accepted material is completely vaporized from an output of said first temperature sensor and said second temperature sensor.

5. The apparatus of claim 1, where said electronics are configured turn the heating element on.

6. The apparatus of claim 5, where said electronics are configured to turn the heating element off when the accepted material is completely vaporized.

7. The apparatus of claim 1, where said apparatus includes a signaling device, and where said electronics are configured to provide an indication of when the accepted material is completely vaporized by activating said signaling device, where said signaling device provides a visual signal, an auditory signal, or a tactile signal.

8. The apparatus of claim 1, where said vaporizer further includes an air inlet to provide air to the receptacle and a mouthpiece to accept air and vapors from the compound and provide the air and vapors to an air outlet, where said electronics a temperature sensor located between said receptacle and an air outlet, and where said indication includes turning off the heater based on an output of said temperature sensor.

9. The apparatus of claim 1, where said heating element is an electrical resistance heating element in contact with the receptacle.

10. An apparatus for vaporizing a compound of a material, said apparatus comprising:
a receptacle for accepting the material;
a heating element adapted to heat the accepted material to a temperature sufficient to vaporize the compound; and
electronics including one or more temperature sensors and power to operate the heating element, where said electronics are configured to turn the heater off when the accepted material is completely vaporized according to the output of at least one of said one or more temperature sensors.

11. The apparatus of claim 10, where said apparatus includes a signaling device, and where said electronics are configured to provide an indication of when the heater is turned off, where said signaling device provides a visual signal, an auditory signal, or a tactile signal.

12. The apparatus of claim 10, where said vaporizer further includes an air inlet to provide air to the receptacle and a mouthpiece to accept air and vapors from the compound and provide the air and vapors to an air outlet, and where said temperature sensor is located between said receptacle and said air outlet.

13. The apparatus of claim 10, where said heating element is an electrical resistance heating element in contact with the receptacle.

14. A method of operating an apparatus for vaporizing a compound of a material, where the apparatus includes a receptacle for accepting the material and a heater to heat the accepted material to a temperature sufficient to vaporize the compound, said method comprising:
estimating if the accepted material is completely vaporized; and
turning off the heater if the accepted material is completely vaporized.

15. The method of claim 14, where the apparatus includes a temperature sensor, and where said estimating estimates using a temperature determined by the temperature sensor.

16. The method of claim 14, where said apparatus includes a first temperature sensor and a second temperature sensor, and where said estimating estimates using an output of the first temperature sensor and an output from the second temperature sensor.

17. The method of claim 14, further comprising:
providing a signal to a user of the apparatus, where said signal is a visual signal, an auditory signal, or a tactile signal.

18. The method of claim 17, where said estimating estimates that the accepted material is completely vaporized when the temperature is equal to or greater than a first temperature

19. A method of operating an apparatus for vaporizing a compound of a material, where the apparatus includes an air inlet to provide air to a receptacle for accepting the material, a heater to heat the accepted material to a temperature sufficient to vaporize the compound, a mouthpiece to accept air and vapors from the receptacle, and a temperature sensor, said method comprising:
turning off the heater when the temperature measured by said temperature sensor equals or is greater than a first temperature, where the first temperature is an indication that the accepted material is completely vaporized.

20. The method of claim 18, further comprising:
providing a signal to a user of the apparatus, where said signal is a visual signal, an auditory signal, or a tactile signal.

21-22. (canceled)
